(19) United States
(12) Patent Application Publication
(10) Pub. No.: US 2005/0203187 A1
(43) Pub. Date: Sep. 15, 2005

(54) Formulations useful for the treatment of varicella zoster virus infections and methods for the use thereof

(76) Inventor: Anthony J. Verbiscar, Sierra Madre, CA (US)

Correspondence Address: FOLEY & LARDNER, P.O. BOX 80278, SAN DIEGO, CA, 92138-0278, US

(21) Appl. No.: 11/064,435

(22) Filed: Feb. 22, 2005

Related U.S. Application Data
(63) Continuation-in-part of application No. 09/795,589, filed on Feb. 28, 2001, now Pat. No. 6,858,232.
Application No. 09/795,589 is a continuation-in-part of application No. 09/320,700, filed on May 26, 1999, now abandoned.
(60) Provisional application No. 60/087,406, filed on Jun. 1, 1998.

(30) Foreign Application Priority Data
May 27, 1999 (WO) PCT/US99/11900

(51) Int. Cl. A61K 31/19; A61K 9/00

(52) U.S. Cl. 514/574; 424/400

(57) ABSTRACT

Jojoba alcohol, a mixture of long chain monounsaturated alcohols, is an oily liquid at moderate ambient temperatures. It is readily absorbed by human skin where it relieves irritation and inhibits the formation of lesions caused by viruses. The inhibitory action is applicable to enveloped viruses which express as sores at dermal surfaces in humans. When applied topically to an incipent herpes episode, it will quickly penetrate the epidermis to the subdermal vascular cells and suppress viral replication which leads to inflammation and the formation of blisters on the face, genital and other skin and mucosal areas. Fumaric acid and malonic acid at low concentrations also inhibit the replication of varicella zoster virus in human cell cultures, with no cellular toxicity. Compositions of certain low molecular weight organic acids in jojoba alcohol enhance antiviral activity. Topical treatment of shingles with a low concentration of fumaric acid in jojoba alcohol terminates the episode. This combination drug acts by a dual mechanism wherein the jojoba alcohol blocks viral fusion by a lipoidal mode, and the polycarboxylic acids inhibit viral fusion by an ionic mode. The combination drug can also be effective in treating chicken pox. Jojoba alcohol is a carrier and transdermal delivery system for these and other pharmacologically active agents for the relief of pain and treatment of other conditions which occur at or under the surface of the skin. Topically applied jojoba alcohol is non-toxic and safe for animals and humans.

### RELATED APPLICATIONS

This application is a continuation-in-part of U.S. application Ser. No. 09/785,589, filed Feb. 28, 2001, now pending, which is, in turn, a continuation-in-part of U.S. application Ser. No. 09/320,700, filed May 26, 1999, now abandoned, which claims priority to Provisional Application No. 60/087,406, filed Jun. 1, 1998. In addition, the present application claims priority from international application No. PCT/US99/11900, filed on May 27, 1999. The entire contents of each of the above-referenced applications are hereby incorporated by reference herein in their entirety.

### FIELD OF INVENTION

This invention relates to methods for the use of liquid mixtures of long chain monounsaturated alcohols such as jojoba alcohol and their compositions for the topical transdermal treatment of subdermal infections caused by such agents as herpes simplex viruses, and the local dermal delivery of pharmacological agents for the treatment of various diseases and other conditions, such as pain. In another aspect, the present invention relates to methods for inhibiting the replication of varicella zoster virus, also known as human herpes virus-3, which is the causative agent for chicken pox and shingles.

### BACKGROUND OF INVENTION

A large percentage of the world population is infected with herpes viruses. Three of the most common herpes viruses are herpes simplex virus-1 (HSV-1), which is the cause of facial and ocular sores, herpes simplex virus-2 (HSV-2), which has a predilection for genital areas, and varicella zoster virus, also named human herpes virus-3 (HHV-3), which causes chicken pox and later shingles. Human herpes virus-8 (HHV-8) is associated with the skin cancer Kaposi sarcoma. These herpes episodes are each susceptible to topical treatments because the viruses replicate in subdermal cells during a recurrence leading to eruption into a lesion.

Once an individual is infected, herpes viruses become latent, principally in nerve cells, and can reactivate to cause recurrences of the original symptoms. When a herpes virus infected individual undergoes stress from exhaustion, strong sunlight, wind, certain foods and medications, menses or microbial infection, the virus migrates to vascular cells under the epidermis where it begins to replicate. For HSV-1 and HSV-2, the initial itchiness, tingling or pain is referred to as the prodromal stage, signaling that the virus is active under the skin. Prodromal can occur from an hour to several days before an outbreak of lesions. At the erythema or inflammation stage, the immune system has begun to fight the virus. After this irritating redness stage, vesicles form and eventually erupt into lesions on the skin and mucosal surfaces. HSV-1 and HSV-2 are morphologically indistinguishable, the main difference being where the sores appear on the skin, and there is some interchange in recurrence sites between these two viruses. Herpes migration to the brain or spinal cord leads to encephalitis and meningitis, which are life-threatening conditions.

Shingles is an infection caused by the varicella zoster virus (VZV), which had become latent following a human episode of chicken pox. Chicken pox affects about 4 million individuals annually, mainly under the age of 18. According to the Center For Disease Control and Prevention, each year in the U.S. chicken pox results in hospitalization of nearly 10,000 individuals, mostly children, and nearly 100 deaths. Once infected, the varicella zoster virus lies dormant in nerve cells and can reappear as shingles in later life. Over the course of an 85 year lifetime, it has been estimated that one in seven people experience at least one episode of shingles. Shingles is an outbreak of a rash and/or blisters on the skin, normally occurring on one side of the body or clustered on one side of the face. Complications include post-herpetic neuralgia (PHN) which can cause debilitating pain that persists for months or even years after the shingles rash has healed. In addition, complications affecting vision and hearing are possible if shingles appears on the face. Principal treatments for shingles are oral nucleoside analog antiviral drugs. This type of drug is slow to act, and can lead to drug resistance, along with nausea and headaches in some patients.

The VZV Research Foundation estimates that more than 800,000 individuals are affected by shingles each year in the U.S. alone. It is most common in people over the age of 50 years, as well as those subject to medical conditions that weaken their immune systems. These include HIV infection, chemotherapy, radiation therapy, transplant operations and stress among others.

Although there is now a vaccine for chicken pox, shingles cannot be prevented, once VZV is contracted. Antiviral drugs, principally the nucleoside analogs, can lessen the duration of shingles and lower the risk of PHN, especially if taken within three days of the appearance of the rash. Immediate treatment with the oral nucleoside analog drugs can help but they are relatively slow to act and are susceptible to cross resistance. To maximize effectiveness and reduce the pain of PHN, it is best to have a drug that acts quickly. PHN pain is due to nerve damage by the virus so a quick effective treatment to reduce the viral load is essential. Neurological complications can include focal muscle paralysis, myelitis, and meningoencephalitis. Shingles should be treated quickly to avoid these and other neurological complications. An advantage in topical treatments is that the drug is applied to specific areas of infection rather than systemically, thereby reducing dosage, response time and side effects.

While there are several treatment options for herpes infections, there are no cures. Several of the nucleoside analog drugs can be effective if taken prophylactically on a daily basis. They are less effective if administered at the time of the recurrence, either orally or topically. The nucleoside drugs inhibit viral replication by penetrating into the cell and interfering with nucleic acid production. They are not virucidal, and depend on a functional immune system to deactivate any virus present. A number of commercial “cold sore” preparations are available which treat symptoms, but are generally ineffective in preventing the formation of lesions. They contain principally anesthetic, antibacterial, emolient and wound healing compounds which can reduce pain, prevent microbial infection and help dry up the blister. Topical treatments of herpes simplex virus infections have been reviewed (Hamuy and Berman, Europ. J. Dermatol., 8:310-319, 1998; Evans and Tyring, Dermatol Clinic, 16: 409-419, 1998; Syed et al., Clin Drug Invest., 16: 187-191, 1998).

Alcohols with chain lengths of 16 to 20 carbon atoms and 1 to 4 double bonds were found to inhibit herpes simplex and another lipid enveloped viral bacteriophage in cell cultures (Sands et al., Antimicrob. Agents Chemother., 15: 67-73, 1979). These unsaturated alcohols were more potent in vitro than saturated alcohols with shorter chain lengths (Snipes et al., Antimicrob. Agents Chemother., 11: 98-104, 1977). A patent (Rivici et al., U.S. Pat. No. 4,513,008, 1985) describes the inhibition of enveloped viruses, such as herpes, with linear polyunsaturated acids, aldehydes or primary alcohols with chain lengths of 20 to 24 carbons and 5 to 7 double bonds. These reports were followed by the investigation and development of n-docosanol as a topical treatment for herpes infections.

n-Docosanol, also named 1-docosanol and behenyl alcohol, is a straight chain 22 carbon saturated alcohol, which occurs in the bark, flowers and fruit of the tree Pygeum africanum. n-Docosanol is reported to have broad activity in cell culture against lipid enveloped viruses such as herpes (Katz et al., Proc. Nat. Acad. Sci., 88:10825-10829, 1991; Katz et al., Ann. N.Y. Acad. Sci., 724: 472-488, 1994; Pope et al., J. Lipid Res., 37: 2167-2178, 1996; Pope et al., Antiviral Res., 40:85-94, 1998), and also the human inmmunodeficiency virus, HIV (Marcelletti et al., AIDS Research and Human Retroviruses, 12: 71-74, 1996). These studies demonstrate that the antiviral activity of n-docosanol includes inhibition of the process of viral entry into the cell, while being mediated by intracellular metabolic biotransformation of the drug. A series of patents on the composition of mixtures of n-docosanol in formulations that render it useful for topical application supports these published reports (Katz, U.S. Pat. No. 4,874,794, 1989; Katz, U.S. Pat. No. 5,071,879, 1991; Katz, U.S. Pat. No. 5,166,219, 1992; Katz, U.S. Pat. No. 5,194,451, 1993; Katz, U.S. Pat. No. 5,534,554, 1996). n-Docosanol is not virucidal (i.e., deactivating viruses directly), but instead it interferes with viral replication, and depends on a functional immune system to destroy herpes viruses. n-Docosanol is a crystalline waxy solid (i.e., insoluble in water), and therefor needs to be formulated with a non-ionic surfactant and carrier to facilitate dermal penetration and interaction at the target cell level. This limitation was also noted where several other long chain compounds with 18 plus linear carbons (including amides, alkanes, acids and alcohols) needed to be formulated with a surfactant and carrier to facilitate penetration of the epidermis (Katz et al., U.S. Pat. No. 5,534,554, 1996; Katz et al., PCT W098/11887, 1998; Katz et al., U.S. Pat. No. 5,952,392, 1999). The latter patents claim a composition of n-docosanol or other long chain compounds, plus a surfactant and a pharmaceutically acceptable diluent or carrier as the active viral replication inhibitor, rather than suggesting such activity by any of the pure individual compounds alone. The solid long chain alcohols and other components of the Katz compositions would not be expected to penetrate skin layers alone without a carrier.

For example, in a study using 10% n-docosanol suspended in an aqueous system containing a non-ionic surfactant and a carrier, mean healing time of lesions in humans infected with herpes labialis (HSV-1) was shortened (Habbema et al., Acta Derm. Venereol., 76: 479-481, 1996). In addition, a 12% n-docosanol cream was tested as a possible transmission prophylactic of simian immunodeficiency virus (SIV) in rhesus macque monkeys (Miller et al., Antiviral Res., 26: A277, 1995). Intravaginal application before exposure prevented transmission in five of the six monkeys tested. n-Docosanol and other saturated alcohols with chain lengths of 20 to 26 carbons reportedly promote corneal healing due to eye injury (Muller, U.S. Pat. No. 5,214,071, 1993; Muller, U.S. Pat. No. 5,296,514, 1994).

Jojoba oil, obtained from the seeds of the desert shrub Simmonsia chinensis, is a mixture of mono esters composed principally of both long chain monounsaturated alcohols and carboxylic acids (Miwa and Spencer, Proc. Second Int. Conf. on Jojoba and Uses, Ensenada, Baja Calif., Mexico, 229-243, 1976). Jojoba oil has been available commercially for more than twenty years, and several million pounds are used in cosmetic formulations annually. Indeed, jojoba oil is a “generally recognized as safe” (GRAS) product for cosmetic uses throughout the world. A significant characteristic of jojoba oil is its ability to be absorbed quickly by the skin. This ready absorption has been related to the single carbon-carbon double bond occurring in the interior of both the alcohol and carboxylic acid parts of the mono ester molecules. Extensive testing and use of jojoba oil has established that it is completely safe when applied to human skin, or administered orally to mice, rats, marmots and rabbits (Taguchi and Kunimoto, Cosmetics and Toiletries, 92: 53-61, 1977; Clark and Yermanos, Biochem. Biophys. Res. Commun., 102: 1409, 1981; Hamm, J. Food Sci., 49: 417-428, 1984; Verschuren and Nugteren, Food Chem. Toxicol., 27: 45-48, 1989). Humans who have ingested jojoba seeds, which are 50% oil, have not been harmed, although some nausea occurred when as much as 200 grams were eaten. In mice, jojoba oil has functioned as an intestinal lubricant (Verbiscar et al., J. Agric. Food Chem., 28: 571-578, 1980). It is estimated that about 20% of jojoba oil is split by hydrolytic enzymes in the gastrointestinal system, thus producing jojoba alcohol in situ. After dermal absorption, jojoba oil is at least partially metabolized to jojoba alcohol.

Jojoba alcohol, derived from the cosmetic ingredient jojoba oil, is an effective topical treatment for cold sores caused by herpes simplex virus-1, and genital sores caused by herpes simplex virus-2 (Verbiscar, Topical Transdermal Treatments, U.S. Pat. No. 6,858,232, 2005). Varicella zoster virus is an enveloped virus also in the herpes family and is known as human herpes virus-3. Jojoba alcohol has been prepared from jojoba oil by reduction with sodium and alcohol (Molaison et al, J. Amer. Oil Chem. Soc., 36: 379-382, 1959). An improved method for the reduction of the ester group of jojoba oil to produce a mixture of monounsaturated alcohols has been reported (see Verbiscar, U.S. Pat. No. 6,703,052, 2004).

In these reduction reactions, the carboxylic acid part of the ester is converted to its corresponding alcohol, in contrast to chemical hydrolysis where the fatty acids remain intact and must be separated from the alcohols in the mixture. Reduction doubles the amount of jojoba alcohol that can be obtained from jojoba oil. One jojoba alcohol product prepared by reduction was reportedly a mixture of octadec-9-enol, eiocos-11-enol, docos-13-enol and tetracos-15-enol (Taguchi, Proc. Sixth Int. Conf. Jojoba and Its Uses, eds. Wisniak and Zabicki, Ben-Gurian Univ. Negev, Beer-Shiva, Israel, p 371-391, 1984). The actual alcohol composition will vary according to the source of jojoba oil used in the reduction reaction. The relative amounts of individual alcohol components in jojoba alcohol depends on the ester composition of jojoba oil. Plant variety, pollination, soil, climate and other environmental conditions will cause the chemical composition of jojoba oil, and thus jojoba alcohol, to vary.

Jojoba alcohol is reported as a lipstick component along with a large number and variety of carboxylic acids, esters and alcohols with diverse structures and functions (Sato, Lipocolor Composition, U.S. Pat. No. 5,700,453, 1997). It is mentioned as an excipient in a formula with kojic acid (Honda, U.S. Pat. No. 5,750,563, 1998). Koey Perfumery Co., Tokyo, a company that introduced jojoba oil commercially as a cosmetic ingredient, also investigated the safety of jojoba alcohol for cosmetic uses (see Examples 1-4; Taguchi, 1984 ibid). The mouse, rabbit, marmot and human tests conducted with jojoba alcohol confirm that this product is very safe for topical application. Mutagenicity tests were also negative.

There is still a need in the art, however, for effective, fast-acting treatments for viral infections, such as shingles. The present invention addresses this and other needs in the art, as will become apparent upon review of the specification and appended claims.

### SUMMARY OF THE INVENTION

In accordance with the present invention, there are provided treatment methods comprising inhibiting viral fusion with, or entry into, a host cell using a mixture of long chain monounsaturated alcohols (e.g., jojoba alcohol) as the inhibitor. Examples of transdermal delivery of drugs are also described. More than 275 unique chemical compounds and compositions have been reported as skin penetration enhancers for transdermal drug delivery (Osborne and Henke, available on the world wide web at pharmtech.com/technical/osborne/osborne/htm).

In accordance with another aspect of the present invention, there are provided treatment methods comprising inhibiting varicella zoster virus replication by contacting the virus with an effective amount of a low molecular weight polycarboxylic acid. Exemplary low molecular weight polycarboxylic acids contemplated for use in the practice of the present invention include compounds which occur in the tricarboxylic acid cycle, in animal and plant metabolism, and the like. In a presently preferred embodiment of the present invention, one or more low molecular weight polycarboxylic acids are applied topically in combination with a mixture of long chain monounsaturated alcohols.

### DETAILED DESCRIPTION OF THE INVENTION

In tests with mice and guinea pigs it was established that jojoba alcohol is not a virucide nor a microbiocide, which destroy viruses directly. Jojoba alcohol is a virustat which delays viral replication by inhibiting cellular penetration. The nucleoside analog drugs such as acyclovir and penciclovir which are used for oral and topical treatments of viral infections are also virustats, acting by interfering with nucleic acid production inside the infected cell. For virustats to be effective, it is necessary for a functional immune system to respond and destroy the virus. Accordingly, it is important to treat herpes recurrences as soon as possible after the virus becomes active, in order to minimize the viral load to be eliminated by the immune system.

Jojoba alcohol is an oily liquid that is readily absorbed by human skin, leaving no residue nor odor. When applied to an incipient herpes simplex virus recurrence, it quickly penetrates the epidermal layer to the subdermal cells where viral replication leading to symptomatic disease would otherwise occur. Without wishing to be bound by any theory, jojoba alcohol appears to function by inhibiting lipid enveloped viruses from fusing with and entering cells. Irritation is relieved and viral replication is delayed, while the host immune system is alerted to destroy the free virus units. When treated early in the prodrome or even the erythema inflammation stages, herpes blisters do not form or at least are inhibited in persons with functional immune systems. This inhibitory action is applicable to enveloped viruses which express as lesions at epidermal surfaces. Herpes simplex viruses which cause recurrent facial sores (HSV-1) and genital sores (HSV-2), shingles (HHV-3) and Kaposi sarcoma (HHV-8) are exemplary treatment targets. Jojoba alcohol also functions as a transdermal carrier for pharmacologically active agents that act at or under dermal surfaces.

There is no single combination of alcohols, nor a percent range, that defines jojoba alcohol. Mixtures of monounsaturated alcohols can also be prepared from other sources, such as by the reduction of sperm whale oil, a monoester similar to jojoba oil, or even from some plant triglycerides. In addition, a mixture of the alcohol components can be prepared by combining each individual alcohol in any specific amount. A formulated mixture of individual long chain alcohols will act like jojoba alcohol. Jojoba alcohol can actually be comprised of a number of individual principally long chain monounsaturated alcohols depending on the source of the seeds from which jojoba oil is derived. Jojoba alcohol is used here as a generic term representing mixtures of these alcohols which will remain liquefied at ambient temperatures above about three degrees centigrade.

In a presently preferred embodiment of the present invention, jojoba alcohol is prepared by the chemical reduction of jojoba oil (see, for example, Verbiscar, U.S. Pat. No. 6,703,052, 2004). In this process jojoba oil, an ester, is reduced to jojoba alcohol with a metal hydride reducing agent, thereby converting the carboxylic acid components of the ester into alcohols as well as the natural alcohol components. Jojoba alcohol is composed of linear long chain monounsaturated alcohols of principally sixteen to twenty four carbon atoms. A gas chromatograph mass spectroscopy analysis of one highly purified jojoba alcohol identified it as a mixture of 0.73% hexadecenol, 7.16% octadecenol, 65.14% eicosenol, 22.23% docosenol and 2.0% tetracosenol. As a mixture, jojoba alcohol remains an oil at room temperature with a freezing point of 10-13° C. When applied to the skin the oil absorbs quickly, and will also carry other drugs with it across the epidermal barrier. In addition, jojoba alcohol is a viral fusion inhibitor which acts by blocking virus entry into a host cell under the surface of the skin. This mode of action limits the viral load for attack by a functional immune system. Jojoba alcohol is a virustat that is active against herpes simplex virus-1 (herpes labialis) and herpes simplex virus-2 (herpes genitalis) in humans. Jojoba alcohol apparently acts by a lipoidal mechanism, interferring with viral fusion at lipid sites on the host cell wall, and lipid sites on the virus envelope.

The mechanism of action of antiviral drugs is significant to their utility. Most of the antiviral drugs in current use are nucleoside analogs which inhibit viral replication inside of the host cell. When administered, they translocate to the host cell where they must penetrate the cell wall then become phosphorylated before interfering with the polymerase reaction forming a viral nucleic acid. This multifaceted mechanism slows the active process and requires rather large doses of an oral drug in order to achieve and maintain efficacy. Several nucleoside antiviral drugs have been developed into topical formulations but these too are still slow to act, even though they do have the benefit of lowering the effective dose required.

Pure long straight chain monounsaturated alcohols are waxy liquids or low melting solids, but when in a mixture as in jojoba alcohol exist as a colorless, odorless, fluid oil at normal ambient temperatures. A characteristic of jojoba alcohol is that it is readily absorbed by human skin and does not require a carrier or surfactant to facilitate transdermal penetration.

Jojoba alcohol is most effective versus herpes recurrences in the prodrome stage, just as the skin is becoming irritated and inflamed, when the immune system begins its response to the localized infection. Early applications every several hours works best, eliminating itchiness and irritation caused by lysis of infected cells. Jojoba alcohol spread on an irritated area of the skin penetrates quickly and is active without the need for a carrier. Compared to small alcohol molecules such as ethanol and isopropyl alcohol, which evaporate or are carried away into the circulation, jojoba alcohol will remain active under the general area of skin application for an extended period of time. Its insolubility in water and the absence of a surfactant limit its absorption into the circulation and removal from the active subdermal site, thereby enhancing activity.

In accordance with another aspect of the present invention, there are provided methods for inhibiting varicella zoster virus replication. Invention methods comprise contacting the virus with an effective amount of a low molecular weight polycarboxylic acid. Exemplary low molecular weight polycarboxylic acids contemplated for use in the practice of the present invention include compounds which occur in the tricarboxylic acid cycle, in animal and plant metabolism, and the like, as well as derivatives thereof. As used herein, “effective amount” refers to amounts effective for the particular therapeutic goal sought, which will, of course, depend on the severity of the condition being treated, and the weight and general state of the subject. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990, each of which is herein incorporated by reference.

The term “effective amount” as applied to invention formulations, means the quantity necessary to effect the desired therapeutic result, for example, a level effective to treat, cure, or alleviate the symptoms of a disease state for which the therapeutic compound is being administered, or to establish homeostasis. Since individual subjects may present a wide variation in severity of symptoms and each drug or active agent has its unique therapeutic characteristics, the precise mode of administration, dosage employed and treatment protocol for each subject is left to the discretion of the practitioner.

Specific examples of low molecular weight polycarboxylic acids contemplated for use in the practice of the present invention include fumaric acid, malonic acid, succinic acid, oxalacetic acid, DL-tartaric acid, L-tartaric acid, citric acid, isocitric acid, DL-malic acid, L-malic acid, maleic acid, glutaric acid, 2-oxoglutaric acid, and the like, as well as mixtures of any two or more thereof. Presently preferred low molecular weight polycarboxylic acids include fumaric acid and malonic acid.

Thus, in a specific aspect of the present invention, fumaric acid and malonic acid have been found to inhibit replication of varicella zoster virus in human cell cultures. Both of these low molecular weight dicarboxylic acids are crystalline solids. Without wishing to be bound by any theory, these dicarboxylic acids apparently act by an ionic mechanism, where viral fusion is impaired by interaction of the carboxylic acid function with the amine base functions in the cell wall and viral envelope. The amine base would be located in the proteinaceous components of these biological structures. The positive in vitro test results with fumaric acid and malonic acid versus VZV are an excellent predictor of their topical activity against the same virus under the surface of the skin. It is essential that these crystalline natural compounds penetrate the epidermis to the cell site where VZV replication is occurring. Therefore, in a presently preferred embodiment of the present invention, jojoba alcohol is employed as a transdermal carrier, whereby the combination drug acts as a viral fusion inhibitor by a dual mechanism, lipoidal and ionic.

Fumaric acid is a dicarboxylic acid among eight or more polycarboxylic acids in the tricarboxylic acid cycle (TCA). The TCA occurs in every living cell in animals, as well as in most plants, as an energy source for the cell. In addition to energy production, the TCA provides intermediates for biosynthesis of more complex products. It is likely that certain of these polycarboxylic acids may serve as a viral control mechanism. These polycarboxylic acids may function independently as ionic viral fusion inhibitors, controlling viral replication to some extent. The mechanism would only be effective for enveloped viruses which contain a protein component in the envelope.

Fumaric acid (trans 2-butenedioic acid) is found widely in nature, in humans and other mammals, and in plants. Fumaric acid is a key intermediate in the tricarboxylic acid cycle (i.e., the Krebs cycle), and is essential to animal and plant tissue respiration. Fumaric acid is present in the mitochondria of cells, and is involved in energy production, protein biosynthesis and lipid metabolism. Psoriasis cells, however, are deficient in fumaric acid. Fumaric acid is a common acidifier and flavoring agent added to foods including breads, beverages, confectioneries and deserts. In some foods it is used as a substitute for tartaric acid and citric acid, two common natural food additives. A salt form, ferrous fumarate, is a hematinic used as an iron supplement. Fumaric acid is also used as an anionic salt component in certain drugs containing an amine functional group.

Malonic acid (propanedioic acid) is an intermediate metabolite in fatty acid biosynthesis and other biochemical functions. Although it is not used directly in foods, its diethyl malonate ester is a food flavoring agent. Fumaric acid and malonic acid are reported to be inhibitors of herpes simplex virus-1 replication in rabbit kidney cell cultures (Poli et al, Food Chem. 4: 251 (1979)), but not in human cells.

In accordance with still another aspect of the present invention, there are provided formulations comprising a low molecular weight polycarboxylic acid and a liquid comprising one or more principally monounsaturated alcohols containing 14 to 24 carbon atoms, as represented by the formula:
CH₃(CH₂)ₘCH═CH(CH₂)ₙCH₂OH
where m and n are each independently 5 to 13 and the carbon-carbon double bonds are cis or trans.

Exemplary principally monounsaturated alcohols contemplated for use in the practice of the present invention include tetradec-7-enyl alcohol, pentadec-7-enyl alcohol, pentadec-8-enyl alcohol, hexadec-7-enyl alcohol, hexadec-8-enyl alcohol, hexadec-9-enyl alcohol, heptadec-7-enyl alcohol, heptadec-8-enyl alcohol, heptadec-9-enyl alcohol, heptadec-10-enyl alcohol, octadec-7-enyl alcohol, octadec-8-enyl alcohol, octadec-9-enyl alcohol, octadec-10-enyl alcohol, octadec-11-enyl alcohol, nonadec-7-enyl alcohol, nonadec-8-enyl alcohol, nonadec-9-enyl alcohol, nonadec-10-enyl alcohol, nonadec-11-enyl alcohol, nonadec-12-enyl alcohol, eicosa-7-enyl alcohol, eicosa-8-enyl alcohol, eicosa-9-enyl alcohol, eicosadec-10-enyl alcohol, eicosa-11-enyl alcohol, eicosa-12-enyl alcohol, eicosa-13-enyl alcohol, uneicosa-7-enyl alcohol, uneicosa-8-enyl alcohol, uneicosa-9-enyl alcohol, uneicosa-10-enyl alcohol, uneicosa-11-enyl alcohol, uneicosa-12-enyl alcohol, uneicosa-13-enyl alcohol, uneicosa-14-enyl alcohol, doseicosa-7-enyl alcohol, doseicosa-8-enyl alcohol, doseicosa-9-enyl alcohol, doseicosa-10-enyl alcohol, doseicosa-11-enyl alcohol, doseicosa-12-enyl alcohol, doseicosa-13-enyl alcohol, doseicosa-14-enyl alcohol, doseicosa-15-enyl alcohol, triseicosa-8-enyl alcohol, triseicosa-9-enyl alcohol, triseicosa-10-enyl alcohol, triseicosa-11-enyl alcohol, triseicosa-12-enyl alcohol, triseicosa-13-enyl alcohol, triseicosa-14-enyl alcohol, triseicosa-15-enyl alcohol, tetraeicosa-9-enyl alcohol, tetraeicosa-10-enyl alcohol, tetraeicosa-11-enyl alcohol, tetraeicosa-12-enyl alcohol, tetraeicosa-13-enyl alcohol, tetraeicosa-14-enyl alcohol, tetraeicosa-15-enyl alcohol, and the like, as well as mixtures of any two or more thereof.

In a presently preferred embodiment of the present invention, the one or more principally monounsaturated alcohols contemplated for use herein comprise jojoba alcohol produced from jojoba oil. In another presently preferred embodiment of the present invention, the one or more principally monounsaturated alcohols contemplated for use herein comprise sperm whale alcohol produced from sperm whale oil. In yet another presently preferred embodiment of the present invention, the principally monounsaturated alcohol contemplated for use herein is oleyl alcohol.

The low molecular weight polycarboxylic acids contemplated for use in this aspect of the present invention include fumaric acid, malonic acid, succinic acid, oxalacetic acid, DL-tartaric acid, L-tartaric acid, citric acid, isocitric acid, DL-malic acid, L-malic acid, maleic acid, glutaric acid, 2-oxoglutaric acid, and the like, as well as mixtures of any two or more thereof. Presently preferred low molecular weight polycarboxylic acids contemplated for use in this aspect of the present invention include fumaric acid and malonic acid.

Optionally, the above-described formulations may further comprise an effective amount of at least one lower alcohol sufficient to maintain the low molecular weight polycarboxylic acid in solution. Presently preferred lower alcohols contemplated for use herein include ethyl alcohol and isopropyl alcohol.

In a preferred embodiment, alpha d-tocopherol (vitamin E) is added to jojoba alcohol to improve stability against oxidation of the double bonds. Tocopherols are the natural antioxidants which occur in jojoba seeds and in pressed or extracted jojoba oils. In a further embodiment, salicylic acid is added as an antiviral, antiseptic and keratolytic agent. This broadens the efficacy of jojoba alcohol and improves its healing power. Other low molecular weight organic acids such as lactic acid, glycolic acid, pyruvic acid, benzoic acid and acetylsalicylic acid will also enhance antiviral efficacy (Poli et al., Food Chem., 4:251-258, 1979; Brown-Skrobot et al., U.S. Pat. No. 4,975,217, 1990; Primache et al., Microbiologica, 21: 397-401, 1998) when present in jojoba alcohol. Additional food grade low molecular weight di- and tri-carboxylic organic acids which in cell cultures have shown activity against herpes and other viruses include malic acid, fumaric acid, succinic acid, tartaric acid and citric acid, but they are formulated with jojoba alcohol into a lower alcohol to improve solubility.

The structure of the major components of this generic jojoba alcohol mixture and examples of the principal individual monounsaturated alcohols composing jojoba alcohol follows:

CH₃(CH₂)ₘCH═CH(CH₂)ₙCH₂OH

| | m | n |
|---|---|---|
| Hexadec-7-enol | 7 | 5 |
| Octadec-7-enol | 9 | 5 |
| Octadec-9-enol | 7 | 7 |
| Eicos-11-enol | 7 | 9 |
| Docos-13-enol | 7 | 11 |
| Tetracos-15-enol | 7 | 13 |

where the double bond can exist in cis and trans forms and m and n can vary from 5 to 13 carbons.

In accordance with yet another aspect of the present invention, there are provided methods for treating episodes characterized by varicella zoster virus replication. Invention methods comprise topically applying any of the above-described formulations to a subject in need thereof. Typically, invention formulations containing in the range of about 0.05 up to about 1% polycarboxylic acid (in a vehicle comprising one or more principally monounsaturated alcohols, optionally containing one or more low molecular weight alcohols) can be applied dropwise as needed.

In accordance with still another aspect of the present invention, there are provided methods for treating episodes characterized by replication of enveloped viruses. Such methods comprise systemically administering any of the above-described polycarboxylic acid-containing formulations to a subject in need thereof.

As readily recognized by those of skill in the art, invention formulations can be prepared for systemic administration in a variety of ways, e.g., by formulating at least one of the above-described polycarboxylic acid compounds in a pharmaceutically acceptable carrier therefor. Exemplary pharmaceutically acceptable carriers include solids, solutions, emulsions, dispersions, micelles, liposomes, and the like. Optionally, the pharmaceutically acceptable carrier employed herein further comprises an enteric coating.

Pharmaceutically acceptable carriers contemplated for use in the practice of the present invention are those which render invention polycarboxylic acid compounds amenable to oral delivery, transdermal delivery, intravenous delivery, intramuscular delivery, topical delivery, nasal delivery, and the like.

Thus, formulations of the present invention can be used in the form of a solid, a solution, an emulsion, a dispersion, a micelle, a liposome, and the like, wherein the resulting formulation contains one or more of the polycarboxylic acid compounds of the present invention, as an active ingredient, in admixture with an organic or inorganic carrier or excipient suitable for enterable or parenteral applications. The active ingredient may be compounded, for example, with the usual non-toxic, pharmaceutically acceptable carriers for tablets, pellets, capsules, suppositories, solutions, emulsions, suspensions and any other suitable for use. The carriers which can be used include glucose, lactose, gum acacia, gelatin, manitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening, and coloring agents and perfumes may be used. The active polycarboxylic acid compound(s) is (are) included in the formulation in an amount sufficient to produce the desired effect upon the process or disease condition.

Invention formulations containing the active ingredient may be in a form suitable for oral use, for example, as tablets, troches, lozenges, aqueous or oily suspensions, dispersible powders or granules, emulsions, hard or soft capsules, or syrups or elixirs. Formulations intended for oral use may be prepared according to any method known to the art for the manufacture of pharmaceutical compositions and such formulations may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing the active ingredient in admixture with non-toxic pharmaceutically acceptable excipients used may be, for example (1) inert diluents such as calcium carbonate, lactose, calcium phosphate or sodium phosphate; (2) granulating and disintegrating agents such corn starch, potato starch or alginic acid; (3) binding agents such as gum tragacanth, corn starch, gelatin or acacia, and (4) lubricating agents such as magnesium stearate, steric acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by such techniques as those described in U.S. Pat Nos. 4,256,108; 4,160,452; and 4,265,874, to form osmotic therapeutic tablets for controlled release.

In some cases, formulations contemplated for oral use may be in the form of hard gelatin capsules wherein the active ingredient is mixed with inert solid diluent(s), for example, calcium carbonate, calcium phosphate or kaolin. They may also be in the form of soft gelatin capsules wherein the active ingredient is mixed with water or an oil medium, for example, peanut oil, liquid paraffin, or olive oil.

Invention formulations may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids, naturally occurring vegetable oils like sesame oil, coconut oil, peanut oil, cottonseed oil, etc. or synthetic fatty vehicles like ethyl oleate or the like. Buffers, preservatives, antioxidants, and the like can be incorporated as required.

Invention formulations may also be administered in the form of suppositories for rectal administration of the drug. These formulations may be prepared by mixing the active agent with a suitable non-irritating excipient, such as cocoa butter, synthetic glyceride esters of polyethylene glycols, which are solid at ordinary temperatures, but liquefy and/or dissolve in the rectal cavity to release the drug. Since individual subjects may present a wide variation in severity of symptoms and each active agent has its unique therapeutic characteristics, the precise mode of administration and dosage employed for each subject is left to the discretion of the practitioner.

Mouse and Guinea Pig Studies: The HSV-2 infected mouse system used here was designed to discover products which have potential to be used in a single dose prophylactic mode against infection. This is principally the domain of virucides, or microbiocides. Viracol is a virustatic and was only expected to delay infection in the mice, which it did as in Example 5. The guinea pig system of Example 6 extends antiinfection tests to first episodes, which are far more severe than recurrences. A principal reason for this is that the newly infected host's immune defense system is not yet programmed to fight this particular virus. Jojoba alcohol merely delays viral entry into the cell where the virus must penetrate in order to replicate. This delay mechanism relies upon an effective immune system to destroy the virus. Jojoba alcohol can only be used to inhibit recurrences, and is preferably applied several times every few hours after the first feeling of irritation signalling an incipient recurrence. For example, treatment of an infected guinea pig every twelve hours as in Example 6 is inadequate for a virustatic which delays viral replication. The application protocol was too infrequent in an animal with a high dose of inoculum, and an immune defense system not yet alerted to the virus. Tests in humans against recurrences of herpes episodes are the best way to evaluate efficacy.

Human Studies: Preliminary tests of jojoba alcohol against herpes labialis (HSV-1) in human subjects were done with pure distilled product with no additives. In one test a 65 year old female subject who normally experiences about two cold sores per year was exposed to strong sunlight for an extended period of time. One day after this exposure the subject began to experience itching and mild inflammation in her upper lip and nasal (perioral) area. One drop of jojoba alcohol was applied and spread around the irritated area, and this was repeated four hours later. Early symptoms disappeared quickly and no lesions formed. This result was typical of several preliminary tests of jojoba alcohol versus herpes simplex virus-1 episodes in human subjects.

Example 7 describes the protocol used to test jojoba alcohol preparations versus HSV-1 induced facial sores and HSV-2 induced genital sores. Results are presented in Tables 1 and 2. In these Tables, jojoba alcohol is formulated with 0.5-1% alpha-d-tocopherol as an antioxidant, and named Viracol. The composition Viracol Plus contains 2% salicylic acid.

TABLE 1
Human Testing Of Viracol Versus Herpes Labialis (HSV-1)

| | | | Stage | Appl. | Lesion Results | | | | |
|---|---|---|---|---|---|---|---|---|---|
| Subject | Gender | Age | Start | Doses | Days | None | Mild | Mod. | Severe |
| 102 | M | 36 | pro | 9 | 1.5 | | | * | |
| 102 | | | pro | 3 | 1 | * | | | |
| 104 | M | 68 | ery | 8 | 3 | * | | | |
| 104 | | | ery | 3 | 1 | * | | | |
| 104 | | | pro | 3/d | 4 | * | | | |
| 107 | M | 40 | pro | 10/d | 7 | | | | * |
| 107 | | | pro | 10/d | 5 | | | | * |
| 107+ | | | pap | 10/d | 2 | | * | | |
| 115 | F | 50 | pro | 3 | 1 | * | | | |
| 115 | | | pro | 2 | 1 | * | | | |
| 115 | | | pro | 4 | 1 | * | | | |
| 132a | F | 50 | pap | 3/d | 6 | | | | * |
| 132 | | | pap | 10/d | 5 | | | * | |
| 132 | | | pap | 10/d | 7 | | | * | |
| 132+ | | | ery | 4/d | 4 | | * | | |
| 132+ | | | ery | 8 | 2.5 | | * | | |
| 133 | M | 52 | pap | 15 | 5 | | | * | |
| 138 | F | 32 | ery | 3 | 1 | | * | | |
| 140+ | | 45 | ery | 70 | 17 | | | * | |
| 140+ | | | pro | 6 | 2 | * | | | |
| 140+ | | | ery | 10 | 3 | | * | | |
| 141b | F | — | pro | 1 | | * | | | |
| 142 | F | 19 | pro | 6 | 1 | * | | | |
| 145 | F | 33 | pap | — | 8 | | * | | |
| 151 | F | 67 | ery | 3 | 1 | * | | | |
| 151 | | | ery | 5 | 1.5 | * | | | |
| 153 | M | 49 | ery | 5 | 7 | | * | | |
| 153 | | | ery | 4 | 1 | | * | | |
| 174+b | F | — | pro | 6 | 2 | * | | | |
| 180+ | F | 45 | pap | 4 | | * | | | |
| Subjects | F = 10, | | | | | 14 | 8 | 5 | 3 |
| | M = 5 | | | | | | | | |

+= Viracol Plus
pro = prodrome,
ery = erythema,
pap = papule
aSubject never without an outbreak of HSV-1 or HSV-2 in past year
bAlso experiences genital HSV lesions

In Table 1, 15 subjects reported a total of 30 recurrences. In 14 (47%) of these episodes there were no sores at all. Another 8 episodes resulted in mild lesions. Several of the moderate to severe lesion recurrences were treated only after blister formation had already started. Several subjects with severe episodes experienced improved results using Viracol Plus. At least one subject (102) experienced all of the symptoms of a cold sore recurrence at one time, with no warning period, but even he was successful in reducing blister formation. One female subject (132), diagnosed with Crohn's disease, has never been without a cold sore and chronic fatigue for more than one year due to a depressed immune system. After several trials with Viracol she was switched to Viracol Plus with improved results. A male subject (107) who also experienced frequent cold sores was also more successful with Viracol Plus. In Viracol Plus, jojoba alcohol acts as a virustatic, delaying viral entry into the cell, while acting as a transdermal delivery system for the solid salicylic acid which inhibits viral replication by a second mechanism related to an interaction with the glycoprotein in the envelope.

TABLE 2
Human Testing Of Viracol Versus Herpes Genitalis (HSV-2)

| | | | Stage | Appl. | Lesion Results | | | | |
|---|---|---|---|---|---|---|---|---|---|
| Subject | Gender | Age | Start | Doses | Days | None | Mild | Mod. | Severe |
| 109 | M | 55 | ery | 3 | 4 | * | | | |
| 111 | M | 33 | pro | 10 | 3 | * | | | |
| 111 | | | pro | 4 | 2 | * | | | |
| 116 | M | 48 | pro | 3 | — | * | | | |
| 124 | F | 65 | pro | 12 | 4 | | * | | |
| 124 | | | pap | 12 | 6 | | | * | |
| 125 | F | 51 | ery | 4 | 2 | * | | | |
| 127 | M | 50 | ery | 2 | 2 | | * | | |
| 130 | M | 52 | pro | 5 | 2 | * | | | |
| 134 | F | 27 | pro | 10 | — | | * | | |
| 134 | | | pro | 5 | 3 | | * | | |
| 136 | M | 49 | pro | 2 | 4 | | * | | |
| 137 | F | 26 | pro | 3 | 2 | | * | | |
| 137 | | | pro | 2 | 1 | * | | | |
| 139 | F | 36 | pap | 4/d | 8 | | | * | |
| 139 | | | ery | 4/d | 6 | | * | | |
| 141^{b} | F | — | pro | 1 | 11 | | | | * |
| 143 | M | 40 | ery | 4 | 4 | * | | | |
| 143 | | | pro | 12 | — | * | | | |
| 148 | M | 44 | pap | 10 | 13 | | | | * |
| 148+ | | | ery | 10 | 4 | | * | | |
| 148+ | | | pro | 4 | 3 | | * | | |
| 148+^{a} | | | pap | 3a | | | | * | |
| 165 | F | 36 | pro | 5 | | * | | | |
| 165 | | | ery | 5 | | * | | | |
| 173+ | F | 26 | pro | 6 | 2 | * | | | |
| 173+ | | | pro | 9 | 3 | * | | | |
| 173+ | | | pro | 3 | 2 | * | | | |
| 174+^{b} | F | 59 | pap | 16 | 8 | | | * | |
| 174+ | | | ery | 3/d | 5 | * | | | |
| 174+^{c} | | | pro | 3/d | 6 | | | | * |
| Subjects | F = 9, | | | | | 15 | 9 | 4 | 3 |
| | M = 8 | | | | | | | | |

+= Viracol Plus
pro = prodrome,
ery = erythema,
pap = papule
^{a}Ran out of Viracol before the episode ended
^{b}Also experienced facial HSV lesions
^{c}Induced by Staph infection

In Table 2, 17 subjects reported a total of 31 genital herpes recurrences. In 15 (48%) of these episodes, there was complete inhibition of lesion formation. Another 9 episodes resulted in mild lesions. Of the 7 episodes where moderate to severe lesions formed, treatment was initiated in 5 episodes at the papule or blister stage. One of these (141) treated her recurrence only once due to employment conditions. A female subject (174) who experienced both HSV-1 and HSV-2 recurrences had greater success with Viracol Plus.

A twelve year old girl experienced pain which after two days led to a few blisters at her waist. Having had chicken pox as an infant, she was diagnosed as having a shingles episode induced by varicella zoster virus (also referred to as human herpes virus-3). Starting on the third day after initiation of pain, she was treated with Viracol six times per day and with oral famciclovir three times per day. The episode was terminated in seven days total. There was no pain and no additional blister formation after treatment was started with Viracol. This is a dramatic improvement over typical treatments, since shingles episodes can be severe and last for several weeks to a month or more.

Jojoba alcohol formulations can be used to treat both men and women with equal success against incipient dermal sores caused by herpes viruses. Results are best when treatment is started as early as possible at the prodrome or erythema stages. The treatments were well tolerated with no adverse effects. Viracol Plus containing salicylic acid improves overall antiviral activity and healing of any blisters that may form due to inadequate application of the jojoba alcohol product, or due to a depressed immune system. In this formulation, jojoba alcohol facilitates the transdermal penetration of salicylic acid, a crystalline solid, to the viral replication site.

Ibuprofen and ketoprofen are two nonsteroidal antimflammatory drugs that are administered orally for management of pain. Some persons using these medications experience gastrointestinal side effects. Each of these drugs was formulated at a 2-5% level in a mixture comprising one or more principally monounsaturated alcohols (e.g., jojoba alcohol). Both formulations successfully relieved pain when applied topically to skin surfaces over the site of the pain. In several cases, relief lasted for several hours following application of only several drops. There was no residue at the application site. The degree of relief varied at different pain sites with different individuals. In one woman with arthritis, a chronic pain in a thumb was relieved. A young boy was relieved of pain in a sprained tendon in the arch of his foot. Another woman used the ketoprofen product to relieve pain in her forearm muscles strained after working at a computer all day. Diclofenac is another oral pain relief drug that can be formulated with a mixture comprising one or more principally monounsaturated alcohols (e.g., jojoba alcohol) for transdermal local delivery against pain.

While the description above contains specifics, these should not be construed as limitations of the scope of the invention, but rather examples of preferred embodiments. Many more variations are possible for the use of jojoba alcohol and other mixtures of long chain monounsaturated alcohols as transdermal delivery agents. Other applications are numerous with several of these noted below, where jojoba alcohol represents any mixture of long chain monounsaturated alcohols.

Alpha hydroxy acids such as lactic acid and glycolic acid are used extensively in cosmetics to reduce wrinkles, spots and other signs of aging (Kurtzweil, Alpha Hydroxy Acids for Skin Care, FDA/CFAN report, U.S. Food and Drug Administration, Consumer Affairs, March-April, 1998). Mixtures comprising one or more principally monounsaturated alcohols (e.g., jojoba alcohol) modulates penetration and safety aspects of these alpha hydroxy acids.

Capsaicin occurs in various edible peppers. It is used as a topical analgesic in gels and lotions to temporarily relieve minor aches and pains associated with arthritis, strains and sprains. Mixtures comprising one or more principally monounsaturated alcohols (e.g., jojoba alcohol) facilitate transdermal penetration of capsaicin at the site of topical application.

Testosterone is a primary male hormone produced in the testes with effects on muscles, bones and sexual function. Testosterone replacement therapy for men with a deficiency, sometimes referred to as hypoganadism, is currently available in topical patches. A 1% testosterone solution in a liquid comprising one or more principally monounsaturated alcohols (e.g., jojoba alcohol) leaves no residue when applied to a subject's forearm, indicating that transdermal delivery was facilitated. This transdermal delivery technology can also be applied to corresponding estrogenic steroids such as estradiol for women.

Prostaglandin E1, also referred to as alprostadil, occurs in the male reproductive system acting as a peripheral vasodilator to support an erection. Several products containing this hormone are now available commercially, one for injection into the corpus cavernosum and one a urethral suppository. Prostaglandin E1 dissolves readily in a mixture comprising one or more principally monounsaturated alcohols (e.g., jojoba alcohol), and facilitates transdermal delivery of this male hormone when applied topically to a penus. This safe and effective administration mode is an improvement over the current modes of administration of prostaglandin E1. Other delivery system enhancers for this hormone are being investigated (Eisenberg and Samour, U.S. Pat. No. 5,527,797, 1996).

Vitamin A (retinol) and/or vitamin D can be formulated with a mixture comprising one or more principally monounsaturated alcohols (e.g., jojoba alcohol) to treat psoriasis. Fumaric acid formulated in a mixture comprising one or more principally monounsaturated alcohols as described herein (e.g., jojoba alcohol) is also useful for treatment of psoriasis.

Pacitaxel (Taxol), its taxane analogs and other anticancer drugs can be formulated with a mixture comprising one or more principally monounsaturated alcohols (e.g., jojoba alcohol) as a penetration enhancer to treat skin cancers such as melanoma and Kaposi sarcoma.

Minoxidil is an antihypertensive drug currently in use as a hair growth stimulant. This active agent is formulated in propylene glycol as a carrier plus alcohol, presumably to facilitate penetration of the scalp. Both dropper and spray applications are available. Minoxidil can be formulated with mixtures comprising one or more principally monounsaturated alcohols (e.g., jojoba alcohol) as a transdermal penetration enhancer, which may also reduce itching and skin irritation side effects. Isopropyl alcohol or ethyl alcohol can be added to the formulation for application as a spray.

The invention will now be described in greater detail with reference to the following non-limiting examples.

### EXAMPLE 1

### Acute Oral Toxicity in Mice

Sixty inbred mice, 30 each male and female, separated into three groups, were fed jojoba alcohol with a stomach tube in a single dose. The first group received 32 ml/kg (27 g/kg), the second 40 ml/kg (34 g/kg), and the third group received 50 ml/kg (42.5 g/kg). There were no deaths in any group after 7 days, so the oral LD50 value is above 50 ml/kg. The average weight dropped on day 1 but increased normally thereafter. Jojoba alcohol probably acts as an intestinal lubricant similar to jojoba oil, causing a weight change in the first 24 hours due to elimination of nutrients along with the jojoba alcohol in feces. There were no observed anatomical changes. Jojoba alcohol was not orally toxic to mice at these dose levels.

### EXAMPLE 2

### Ocular and Dermal Rabbit Tests

Jojoba alcohol was dissolved in jojoba oil at three concentration levels of 50%, 25% and 12.5% on a w/w basis. The rabbits, three per dose level group, were administered 0.05 ml (1 drop) of these solutions in the right eye. The left eye was not treated. Eye irritation was very low with no effects on the cornea and iris, and mild conjunctivitis clearing up within 24-48 hours. In another test, ten male albino rabbits were treated with cloth strip patches on the skin with each of these three samples. Patches were removed from 5 rabbits after 15 days and from the remaining 5 rabbits after 30 days. Visual and pathological examination of the treated skin areas indicated that irritation was quite low and comparable among the three samples.

### EXAMPLE 3

### Dermal and Subcutaneous Marmot Tests

Jojoba alcohol was dissolved in high purity jojoba oil at a 10% concentration. Albino marmots, 10 males and 10 females, were treated with this sample in a patch test. There was no sign of any irritation after 24 and 48 hours. In another test, the 10% solution of jojoba alcohol in jojoba oil was injected subcutaneously into 10 each male and female marmots. After 24 and 48 hours there was no evidence of irritation at the injection site. After one week the jojoba alcohol solution was spread on a cloth patch, and the patch was placed on the injection site. After two weeks a jojoba alcohol solution sample patch was placed on a challenge site away from the site of injection. No sensitization was observed at any of the sites.

### EXAMPLE 4

### Dermal Patch Human Tests

A test was carried out on 40 humans with healthy skin. Two samples including 100% jojoba alcohol and 10% jojoba alcohol in jojoba oil were prepared on cloth strip patches. The patches were applied on the upper part of the back of 20 subjects for each sample. Results were observed after 30 minutes and after 24 hours. No evidence of irritation of any kind was observed in 39 of the subjects, and only one of the subjects on the 10% formula showed a possible reaction. A second test was carried out on another 40 subjects with contact dermatitis using pure jojoba alcohol on cloth strips patches. Only one of the test subjects showed a doubtful reaction in the first 30 minutes, and there were no positives after 24 hours. Jojoba alcohol is dermally non-toxic.

### EXAMPLE 5

### Prophylactic Test Versus Herpes Simplex Virus-2 in Mice

Thirty Swiss Webster female weanling mice with an average weight of 21 grams were divided into two groups of 15 controls and 15 treatment mice. Immediately before viral inoculation the controls were administered a placebo of phosphate buffer saline and the treatment mice were each administered 15 microliters of jojoba alcohol intravaginally. All 30 mice were then inoculated intravaginally with 10,000 pfu's of strain 186 herpes simplex virus-2 (HSV-2) and then maintained for 21 days. There was no apparent toxicity due to the mode of administration.

Results indicated that jojoba alcohol delayed the effects of HSV-2 infection in mice, namely death, but did not significantly prevent infection. On day 15 post inoculation, 80% of placebo mice had died compared to 40% of the treatment group. This effect could have resulted from limiting the initial viral replication in the genital tract, and thus decreasing the quantity of virus reaching latent storage sites in the ganglia. However, by day 21 only one treatment mouse survived and all placebo mice had died. This test clearly demonstrates that viracol is not virucidal and should not be used as a prophylactic to prevent transmission of HSV-2.

### EXAMPLE 6

### First Episode Test Versus Herpes Simplex Virus-2 in Guinea Pigs

Twenty four Hartley female guinea pigs weighing 300-350 grams were divided into two groups of 12 each, receiving jojoba alcohol and no treatment. Treatment animals received 0.05 ml (1 drop) of jojoba alcohol intravaginally, immediately followed in both groups by intravaginal inoculation of 75,000 pfu's of herpes simplex virus-2, a very high viral load which assures establishment of viral infection. Subsequent intravaginal/topical treatments with 0.05 mls of jojoba alcohol were applied 12 hours post viral inoculation, and continued twice daily every 12 hours for the following seven days. All animals were examined daily for evidence of primary episode herpetic disease, which began to appear on day 3 and continued for as long as day 10. Two treatment animals and one control remained asymptomatic. Jojoba alcohol did not significantly reduce the incidence or severity of this primary episode in guinea pigs, but there were no side effects due to intravaginal administration.

### EXAMPLE 7

### Human Studies Versus Herpes Simplex Viruses

A clinical study was undertaken using pure jojoba alcohol, a solution of 1% alpha-tocopherol in jojoba alcohol named Viracol, and a solution of 2% salicylic acid in Viracol named Viracol Plus. Male and female subjects of any age were enrolled who experienced at least 2 or 3 recurrences of herpes labialis (HSV-1) or herpes genitalis (HSV-2) per year. Exclusions included pregnant women, subjects on chronic antiviral chemotherapy, immunotherapy or alternative therapy, and hypersensitive individuals. Subjects were instructed to apply the jojoba alcohol products 3 to 5 times every 2 to 3 hours as soon as possible after initial irritation. They were provided with several reporting cards which included entries for the subject's code number, herpes virus identity, treatment date, number of applications, stage application started (prodrome, erythema, papule), lesions (none, mild, moderate, severe), total days of episode and side effects/comments. All subjects were volunteers, as were all herpes episodes meaning no recurrences were induced. Subjects were on an honor system to fill out the report cards after treating a recurrence, and mailing them back to the laboratory. Results are summarized in Table 1 and Table 2.

### EXAMPLE 8

### Nonsteroidal Antiinflammatory Agents in Jojoba Alcohol

Ibuprofen, alpha-methyl-4(2-methylpropyl)benzeneacetic acid, is an orally active antiinflammatory agent. It was dissolved at a 5% level in jojoba alcohol containing 1% alpha-d-tocopherol as an antioxident. The oil was tested on several persons for relief of minor muscle and joint pains. Several drops of this preparation absorbs readily into human skin tissue with no visible residue, while relieving pain.

Ketoprofen, 3-benzoyl-alpha-methylbenzeneacetic acid, is an orally active antiinflammatory and analgesic agent. It was disolved at a 3% level in jojoba alcohol containing 1% alpha-d-tocopherol as an antioxident. The oil was tested topically on several persons for relief of minor muscle and joint pains. Several drops of this preparation absorbs readily into human skin tissue with no visible residue, while relieving pain.

### EXAMPLE 9

### Low Molecular Weight Organic Acids in Jojoba Alcohol

Salicylic Acid is an antiviral, antiseptic and keratolytic agent. It was dissolved at a 2% level in jojoba alcohol. This beta-hydroxy acid enhances the efficacy of jojoba alcohol as an antiviral. It also improves healing power when applied at a papule stage of a herpes recurrence.

Acetylsalicylic Acid (aspirin) is an alalgesic, antipyretic and antiinflammatory agent that is also active against herpes simplex virus-3 (varicella zoster), the cause of chicken pox and shingles. Acetylsalicylic acid dissolves readily in jojoba alcohol.

Benzoic Acid is a food additive functioning as a preservative. Benzoic acid has germicidal activity and dissolves readily in jojoba alcohol.

Lactic Acid occurs naturally in yogurts and sour milk and is an acidulent used in foods. A 5% solution in jojoba alcohol absorbed readily into human skin with no irritation. Lactic acid is used in cosmetics, and has shown viral replication inhibition activity in cell cultures.

Glycolic Acid occurs in sugar cane juice. It dissolves readily in jojobal alcohol and is used in cosmetics. Glycolic acid has shown viral replication inhibition activity in cell cultures.

Pyruvic Acid is a natural component of muscle metabolism. Pyruvic acid dissolves readily in jojoba alcohol, and has shown viral replication inhibition activity in cell cultures.

### EXAMPLE 10

### Di- and Tri-Carboxylic Organic Acids in Jojoba Alcohol Lotions

Malic Acid also known as hydroxysuccinic acid, occurs naturally in apples and is sometimes referred to as apple acid. It is used as a general purpose acidulent in food products. A 40 mg quantity dissolved readily in 3 ml of ethyl alcohol and 3 ml of jojoba alcohol resulting in a lotion containing 0.8% malic acid. This lotion applied to human skin absorbed readily and was non-irritating, leaving no residue. Malic acid has shown viral replication inhibition activity in cell cultures.

Citric Acid occurs in citrus and many other fruits, and is widely distributed in animal tissues. It is a sequestrant food additive and one of the active ingredients in Alka-Seltzer. A 35 mg quantity dissolved readily in 4 ml of isopropyl alcohol and 4 ml of jojoba alcohol resulting in a lotion containing 0.5% citric acid This lotion absorbed readily in human skin and was non-irritating, leaving no residue. Citric acid has shown viral replication inhibition activity in cell cultures.

Fumaric Acid is essential to animal tissue respiration. It is used as a substitute or partial replacement for tarric acid or citric acid in beverages. It can be formulated with jojoba alcohol and a lower alcohol such as ethyl alcohol and isopropyl alcohol as a lotion. Fumaric acid has shown viral replication inhibition in cell cultures.

Succinic Acid is used as a buffer and neutralizing agent in foods. It can be formulated with jojoba alcohol and a lower alcohol such as ethyl alcohol or isopropyl alcohol as a lotion. Succinic acid has shown viral replication inhibition activity in cell cultures.

### EXAMPLE 11

### Evaluation of Jojoba Alcohol in a Shingles Episode

A twelve year old girl experienced pain which after two days led to blisters at her waist, and was diagnosed as having a shingles episode. Starting on the third day after initiation of pain, she was treated topically with jojoba alcohol six times per day and with oral famcyclovir three times per day. The episode terminated in seven days total. There was no additional blister formation after treatment was started with jojoba alcohol. This incident indicates that treatment with jojoba alcohol can relieve itching and pain, inhibit blister formation and shorten healing time for a shingles episode.

### EXAMPLE 12

### Evaluation of Antiviral Activity in Human Cell Cultures

Human foreskin fibroblast (HFF) cells obtained from newborns were prepared and suspended in minimum essential medium (MEM) and supplemented with 10% fetal bovine serum (FBS). Freshly processed cells were incubated for 24 hours at 37° C. in well tissue culture plates in a CO₂ incubator (E. R. Kern, J. Infect. Dis., 128:290 (1973)). Assays for cytotoxicity and varicella zoster virus replication were carried out in these culture wells following reported procedures (see, for example, R. J. Ryback and E. R. Kern et al, Antimicrobial Agents and Chemotherapy, 44:1506 (2000); Y. L. Qiu et al, J. Med. Chem., 41: 10 (1998); and Y. L. Qiu et al, J. Med. Chem., 41: 5257 (1998)). Drug concentrations were diluted six times from 100 μg/ml down to 0.03 μg/ml in MEM/FBS solution. A selective index of 10 is considered significant. Results are presented in Table 3.

TABLE 3
Varicella Zoster Virus in Cultures of Human Foreskin Fibroblast Cells

| | EC50 μg/ml | EC90 μg/ml | CC50 μg/ml | SI |
|---|---|---|---|---|
| Malonic acid | <0.03 | <0.03 | >100 | >3333 |
| Fumaric acid | 0.15 | 0.6 | >100 | >666 |
| Acyclovir | 0.04 | 0.09 | >100 | >2500 |

Acyclovir positive antiviral control drug
EC50 effective concentration required to inhibit viral replication by 50%
EC90 effective concentration required to inhibit viral replication by 90%
CC50 cytotoxicity concentration required to inhibit stationary cells by 50%
SI selective index of CC50/EC50

### EXAMPLE 13

### Evaluation of Antiviral Activity in a Shingles Episode

A 47 year old man who had chicken pox as a child experienced a recurrence as shingles. This emerged as a 2×15 inch band of inflammation, blisters and pain around his front waist. After 6-8 months a 2 inch diameter outbreak of blisters occurred on his arm above the fold at his elbow, and he was started on oral acyclovir at 400 mg 2×/day. After several days there was no change in his shingles, although he developed diarrhea and headache side effects from this medication. In view of the lack of effectiveness of traditional methods of treatment, the subject agreed to try the above-described Viracol A Plus composition on his shingles.

The Viracol A Plus formulation contained 90% Viracol (i.e., jojoba alcohol+0.5% alpha-d-tocopherol), 10% ethanol and 0.2% w/v fumaric acid. A test of one drop on the subject's arm showed that it absorbed quickly with no irritation. For the next 8 days he spread 1-2 drops twice each day on the 2.5 inch diameter outbreak of small blisters on his arm, and 4-6 drops twice each day on the blisters at his waist. Itching at both locations quickly terminated after each treatment. This indicated that viral replication in cells with their subsequent disruption was inhibited. This did not occur with acyclovir alone. At 8 days of Viracol A Plus treatment the blisters on his arm had disappeared completely, and his waist was healing with a few small residual sores and some inflammation. Some neuralgia in his arm remained but the pain in his waist subsided substantially. The subject continued on his 3 remaining tablets of acyclovir at 1/day, and continued to use Viracol A Plus on his waist until it too healed completely within two weeks.

The results provided herein demonstrate that jojoba alcohol acts as a carrier for dicarboxylic acids, as well as being a viral fusion inhibitor, presumably by a lipoidal mode of action. The jojoba alcohol/fumaric acid formulation, termed Viracol A Plus, absorbs quickly into the skin with no residue nor odor. This dual action formulation is an effective way to treat varicella zoster virus infection (shingles), and is expected to also be active in a chicken pox episode. Viracol A Plus inhibits viral replication and reduces itching and PHN pain due to nerve cell damage.

While the invention has been described in detail with reference to certain preferred embodiments thereof, it will be understood that modifications and variations are within the spirit and scope of that which is described and claimed.

1. A method for inhibiting varicella zoster virus replication, said method comprising contacting said virus with an effective amount of a low molecular weight polycarboxylic acid.

2. A method according to claim 1 wherein said low molecular weight polycarboxylic acid is selected from the group consisting of fumaric acid, malonic acid, succinic acid, oxalacetic acid, DL-tartaric acid, L-tartaric acid, citric acid, isocitric acid, DL-malic acid, L-malic acid, maleic acid, glutaric acid, 2-oxoglutaric acid and mixtures of any two or more thereof.

3. A method according to claim 1 wherein said low molecular weight polycarboxylic acid is fumaric acid.

4. A method according to claim 1 wherein said low molecular weight polycarboxylic acid is malonic acid.

5. A formulation comprising a low molecular weight polycarboxylic acid and a liquid comprising one or more principally monounsaturated alcohols containing 14 to 24 carbon atoms, as represented by the formula:
CH₃(CH₂)ₘCH═CH(CH₂)ₙCH₂OH
where m and n are each independently 5 to 13 and the carbon-carbon double bonds are cis or trans.

6. A formulation according to claim 5 wherein the one or more principally monounsaturated alcohols is selected from the group consisting of tetradec-7-enyl alcohol, pentadec-7-enyl alcohol, pentadec-8-enyl alcohol, hexadec-7-enyl alcohol, hexadec-8-enyl alcohol, hexadec-9-enyl alcohol, heptadec-7-enyl alcohol, heptadec-8-enyl alcohol, heptadec-9-enyl alcohol, heptadec-10-enyl alcohol, octadec-7-enyl alcohol, octadec-8-enyl alcohol, octadec-9-enyl alcohol, octadec-10-enyl alcohol, octadec-11-enyl alcohol, nonadec-7-enyl alcohol, nonadec-8-enyl alcohol, nonadec-9-enyl alcohol, nonadec-10-enyl alcohol, nonadec-11-enyl alcohol, nonadec-12-enyl alcohol, eicosa-7-enyl alcohol, eicosa-8-enyl alcohol, eicosa-9-enyl alcohol, eicosadec-10-enyl alcohol, eicosa-11-enyl alcohol, eicosa-12-enyl alcohol, eicosa-13-enyl alcohol, uneicosa-7-enyl alcohol, uneicosa-8-enyl alcohol, uneicosa-9-enyl alcohol, uneicosa-10-enyl alcohol, uneicosa-11-enyl alcohol, uneicosa-12-enyl alcohol, uneicosa-13-enyl alcohol, uneicosa-14-enyl alcohol, doseicosa-7-enyl alcohol, doseicosa-8-enyl alcohol, doseicosa-9-enyl alcohol, doseicosa-10-enyl alcohol, doseicosa-11-enyl alcohol, doseicosa-12-enyl alcohol, doseicosa-13-enyl alcohol, doseicosa-14-enyl alcohol, doseicosa-15-enyl alcohol, triseicosa-8-enyl alcohol, triseicosa-9-enyl alcohol, triseicosa-10-enyl alcohol, triseicosa-11-enyl alcohol, triseicosa-12-enyl alcohol, triseicosa-13-enyl alcohol, triseicosa-14-enyl alcohol, triseicosa-15-enyl alcohol, tetraeicosa-9-enyl alcohol, tetraeicosa-10-enyl alcohol, tetraeicosa-11-enyl alcohol, tetraeicosa-12-enyl alcohol, tetraeicosa-13-enyl alcohol, tetraeicosa-14-enyl alcohol, tetraeicosa-15-enyl alcohol, and mixtures of any two or more thereof.

7. A formulation according to claim 5 wherein the one or more principally monounsaturated alcohols comprises jojoba alcohol produced from jojoba oil.

8. A formulation according to claim 5 wherein the one or more principally monounsaturated alcohols comprises sperm whale alcohol produced from sperm whale oil.

9. A formulation according to claim 5 wherein said principally monounsaturated alcohol is oleyl alcohol.

10. A formulation according to claim 5 wherein said low molecular weight polycarboxylic acid is selected from the group consisting of fumaric acid, malonic acid, succinic acid, oxalacetic acid, DL-tartaric acid, L-tartaric acid, citric acid, isocitric acid, DL-malic acid, L-malic acid, maleic acid, glutaric acid, 2-oxoglutaric acid and mixtures of any two or more thereof.

11. A formulation according to claim 5 wherein said low molecular weight polycarboxylic acid is fumaric acid.

12. A formulation according to claim 5 wherein said low molecular weight polycarboxylic acid is malonic acid.

13. A formulation according to claim 5, further comprising an effective amount of at least one lower alcohol sufficient to maintain said low molecular weight polycarboxylic acid in solution.

14. A formulation according to claim 13 wherein said lower alcohol is ethyl alcohol or isopropyl alcohol.

15. A method for treating episodes characterized by varicella zoster virus replication, said method comprising topically applying a formulation according to claim 5 to a subject in need thereof.

16. A method for treating episodes characterized by varicella zoster virus replication, said method comprising topically applying a formulation according to claim 6 to a subject in need thereof.

17. A method for treating episodes characterized by varicella zoster virus replication, said method comprising topically applying a formulation according to claim 7 to a subject in need thereof.

18. A method for treating episodes characterized by varicella zoster virus replication, said method comprising topically applying a formulation according to claim 8 to a subject in need thereof.

19. A method for treating episodes characterized by varicella zoster virus replication, said method comprising topically applying a formulation according to claim 9 to a subject in need thereof.

20. A method for treating episodes characterized by varicella zoster virus replication, said method comprising topically applying a formulation according to claim 10 to a subject in need thereof.

21. A method for treating episodes characterized by varicella zoster virus replication, said method comprising topically applying a formulation according to claim 11 to a subject in need thereof.

22. A method for treating episodes characterized by varicella zoster virus replication, said method comprising topically applying a formulation according to claim 12 to a subject in need thereof.

23. A method for treating episodes characterized by varicella zoster virus replication, said method comprising topically applying a formulation according to claim 13 to a subject in need thereof.

24. A method for treating episodes characterized by varicella zoster virus replication, said method comprising topically applying a formulation according to claim 14 to a subject in need thereof.

25. A method for treating episodes characterized by replication of enveloped viruses, said method comprising systemically administering a formulation containing one or more of the polycarboxylic acids in claim 2.
